(19) United States
(12) Patent Application Publication
(10) Pub. No.: US 2024/0377390 A1
(43) Pub. Date: Nov. 14, 2024

(54) SYSTEMS AND METHODS FOR CYTOCAPTURE OF IMMUNE CELLS TO DETECT INTERNAL INFECTIONS

(71) Applicant: The George Washington University, Washington, DC (US)

(72) Inventor: Timothy A. McCaffrey, Washington, DC (US)

(21) Appl. No.: 18/683,196

(22) PCT Filed: Sep. 9, 2022
(86) PCT No.: PCT/US2022/076205

§ 371 Date: Feb. 12, 2024

(51) Int. Cl. G01N 33/543; G01N 21/59

(52) U.S. Cl. CPC G01N 33/54326; G01N 21/59; G01N 2333/70596; G01N 2333/96441

(57) ABSTRACT

The present technology detects immune cell activation. In certain embodiments, the system has a polymer block with a reaction chamber having an optically clear light path and fluidic connections to a device having a microcontroller in communication with electromagnets, air pumps, valves, an LED light source, and a photosensor. The polymer block is able to receive a blood sample in media comprising antibody coated beads.

### CROSS-REFERENCE TO RELATED APPLICATIONS

This application claims the benefit of U.S. Provisional App. No. 63/242,219, filed Sep. 9, 2021, the entire contents of which are incorporated herein by reference.

### BACKGROUND

Physicians lack adequate information to distinguish internal infections by bacteria, viruses, or other pathogens, from sterile inflammatory or neurogenic pain disorders. Existing tests, such as an elevated white blood cell (WBC) count, are relatively insensitive, and non-specific for detecting infections. Existing tests for infections, such as the WBC count, take a large volume of blood (˜3 mls) via venopuncture, which is sent to an in-hospital lab, or to an external lab service, requiring between hours and days for results. Private physicians typically must refer the patient to a separate lab for a complete blood count (CBC) blood draw. In addition to the limitations of this methodology in standard clinical care, this inaccurate and inefficient method for infection detection creates a large strategic gap in US Health Security. By the time a biothreat is identified and a diagnostic test is deployed, a pandemic is well underway. As noted below, the methods known in the art are deficient, and inferior to the present technology.

It is known that the innate immune system, which includes specialized white blood cells called polymorphonuclear neutrophils (PMNs), reacts to infections by altering the RNA and protein content of the immune cells, especially in the PMNs. This cellular response by immune cells creates the opportunity to identify circulating biomarkers of internal infections. However, there are at least 3 major problems with existing strategies to identify and employ circulating biomarkers of infection:

(1) The major problem with many biomarkers is that while they are elevated in circulating cells, it is difficult to measure them in plasma or serum because they are not released from the cell.

(2) Even if immune cells do release immune mediators into plasma, they are rapidly inactivated in plasma by known inhibitors. It is a physiological necessity that reactive inflammatory markers must be tightly controlled in time and space to prevent local inflammation from becoming systemic inflammation.

(3) In the process of making plasma or serum via centrifugation, those biomarkers can be released non-specifically from cells, thereby creating a ‘false positive’ signal.

We can illustrate these problems by using one particular biomarker that will be a focus of the present invention, neutrophil elastase (NE), that is typically stored in PMN granules. Examples of prior attempts to use neutrophil elastase as a biomarker for disease includes the following:

Zeming describes a microfluidic device that profiles enzymatic activity in immune cells isolated from blood. However, there are 4 notable differences: a) it utilizes physical isolation of leucocytes from blood based on physical properties, (vs surface antigens in the present invention), b) it offers no specificity for the type of leucocyte isolated (vs PMNs in the present invention), and c) it is explicitly intended for ‘secreted’ enyzmes, and thus uses droplet encapsulation and extracellular substrates (vs cellular elastase activity in the present invention), and d) it is explicitly intended for measuring these secreted enzymes in relation to heart failure (vs internal infection detection in the present invention). No data regarding the detection of internal infections is provided by this publication. Zeming K K, Lu R, Woo K L, Sun G, Quek K Y, Cheow L F, et al. Multiplexed Single-Cell Leukocyte Enzymatic Secretion Profiling from Whole Blood Reveals Patient-Specific Immune Signature. Anal Chem. 2021; 93 (10): 4374-82.

Jundi describes a microfluidic device that isolates leucocytes for sensing the severity of sepsis. There are several notable differences: a) the method uses centrifugal forces that at best can separate leucocytes from red blood cells (RBC) and platelets, but has no specificity for PMNs, b) their own data shows that the efficiency is markedly lower than density based methods, and does not address or compare to magnetic separation, c) the functional assessment of the centrifugally isolated total leucocytes is based on triggered release of enzymatic activity (including elastase), vs the endogenous cellular elastase activity in the present example, and d) sepsis is different than internal infection: very few internal infections lead to sepsis, which is an exaggerated response to an infection. While they make some reference to PMNs, this is based on secondary flow cytometric purification and quantitation, not within their device. Furthermore, their surface marker is CD16, not CD15, which identifies a somewhat different cell type, and most importantly, they did not observe a significant positive relationship between the number of these CD16+ cells or their triggered elastase release, and the severity of sepsis. In short, this paper does not teach that you could diagnose the presence of internal infection from PMN elastase activity. Jundi B, Ryu H, Lee D-H, Abdulnour R-E E, Engstrom B D, Duvall M G, et al., Leukocyte function assessed via serial microlitre sampling of peripheral blood from sepsis patients correlates with disease severity, Nat Biomed Eng., 2019, 3 (12): 961-973.

Shoemark describes a point of care (POC) device for measuring neutrophil elastase antigen for the purpose of determining bronchiectasis (airway dilatation) severity, airway infection, and risk of exacerbation. However, this device is very different from the present invention: a) it utilizes sputum, b) it measured free, not cellular elastase, c) it measured elastase protein antigen by a commercially available lateral flow device, and d) it does not use cell enrichment. In short, this is an entirely different method compared to the present invention. Shoemark A, Cant E, Carreto L, Smith A, Oriano M, Keir H R, et al., A point-of-care neutrophil elastase activity assay identifies bronchiectasis severity, airway infection and risk of exacerbation, Eur Respir J. 2019, 53 (6): 1900303.

Plasma neutrophil elastase (NE) protein antigen, either in sputum or plasma, was not statistically different between patients with cystic fibrosis (CF, n=16) and CF patients with a bacterial infection. A. AbdulWahab, M. Allangawi, M. Thomas, I. Bettahi, S. K. Sivaraman, J. Jerobin, et al. Sputum and Plasma Neutrophil Elastase in Stable Adult Patients With Cystic Fibrosis in Relation to Chronic Pseudomonas Aeruginosa Colonization, Cureus 2021, 13(6):e15948.

Ng et al. reported modest, but statistically significant increases in plasma elastase antigen in COVID-19 patients, but do not describe the diagnostic value in detecting viral or bacterial infection. In this study, the NE was measured on plasma samples by well known antibody-based laboratory methods that would be impractical in routine clinical use. Ng H, Havervall S, Rosell A, Aguilera K, Parv K, von Meijenfeldt F A, et al., Circulating Markers of Neutrophil Extracellular Traps Are of Prognostic Value in Patients With COVID-19, Arterioscier Thromb Vasc Biol. 2021, 41(2):988-94.

Elastase protein antigen, measured as a complex with its natural inhibitor, alpha1-antitrypsin (α1AT), did not improve the diagnostic utility of other markers in discriminating Kawasaki disease, an inflammatory vascular disease, from infection in children. Zandstra J, van de Geer A, Tanck M W T, van Stijn-Bringas Dimitriades D, Aarts C E M, Dietz S M, et al., Biomarkers for the Discrimination of Acute Kawasaki Disease From Infections in Childhood, Front Pediatr. 2020, 8:355.

Serum elastase protein antigen did not provide statistically significant differences between subjects with or without infections due to pacemaker placement. Lennerz C, Vrazic H, Haller B, Braun S, Petzold T, Ott I, et al. Biomarker-based diagnosis of pacemaker and implantable cardioverter defibrillator pocket infections: A prospective, multicentre, case-control evaluation. PloS one. 2017; 12(3):e0172384. The papers mentioned herein are incorporated by reference.

These prior approaches have attempted to use NE protein antigen in plasma as a biomarker of internal disease, but have not met with clinical success for the 3 main reasons discussed above: 1) NE is not secreted until a late stage of PMN activation, 2) NE proteins are rapidly bound, inactivated, and cleared from the circulation, and 3) NE antigen can be non-specifically released in the process of blood sampling and plasma purification. The overwhelming effect of serine protease inhibitors (SERPINs) in plasma likely complicates the utility of this approach. However, the following examples demonstrate that if PMNs are isolated from whole blood prior to the quantitation of NE activity by a functional approach, the NE activity can be diagnostically useful. Further, the present invention provides for a method and device to measure the PMN elastase activity rapidly at the point-of-care (POC), thus increasing its utility greatly.

### SUMMARY OF THE INVENTION

The disclosed embodiments provide systems and methods for a point-of-care (POC) test to capture human immune cells and quantify specific functional biomarkers of viral and/or bacterial infection. Termed ‘CytoCapture of Biomarkers In Situ (CyBIS)’, the method is a unique approach that avoids numerous problematic steps in the detection of mammalian pathogens via host biomarkers. Briefly, CyBIS is a method implemented on a POC device that measures blood polymorphonuclear leucocytes (PMN, neutrophil) number and activation level as an indicator of an internal infection. CyBIS results reflect the number of circulating PMNs, which is a known metric of infection (albeit not at POC), and adds the novel dimension that the elastase activity per PMN is markedly increased. One use of the CyBIS test is the detection of unknown/variant pathogen infections via host immune activation. A second use addresses a vast unmet medical need for a simple test to quickly distinguish bacterial, viral, or fungal infections from sterile inflammation or physical trauma. CyBIS, combined with other clinical parameters, provides actionable information to physicians such that if the CyBIS score is elevated outside of the normal range, then the physician can consider prescribing antibiotics, order further imaging studies, obtain a sample of the suspected infection for laboratory analysis, or, in severe cases, to consider surgical removal of the infected tissues or prosthetic in the body. If CyBIS scores are in the normal range for subjects without infections, then this informs the physician to search for non-infectious causes. Non-infectious causes of abdominal pain can include torsion (twisting) of the intestines or Fallopian tubes, or blockage of the intestines, Fallopian tubes, or urogenital canals. In such cases, the physician may elect some type of surgical correction. Other non-infectious causes of pain can be autoimmune attack on the intestines or other internal organs, and in such cases the physician may elect to prescribe anti-inflammatory medications. Another common, but non-infectious cause of abdominal pain would be some type of physical trauma to the abdomen, and in such cases, the physician may elect to repair it surgically, or allow it to heal, while providing some type of pain relief medication.

In certain embodiments, the technology comprises methods for the purification of neutrophils from whole blood, by a rapid but gentle method, and measurement of neutrophil activation markers (i.e., neutrophil elastase), from which neutrophil elastase activity can be observed as a highly accurate biomarker of an infection in a subject.

In other embodiments, the technology comprises methods and devices that can conduct this isolation and elastase assay on a small volume of blood in approximately 30 minutes.

In yet other embodiments, the technology comprises the isolation of PMNs in a non-destructive manner, combined with the ability to measure their elastase activity accurately in a kinetic assay.

The POC CyBIS device, as presently rendered to use, is acceptable for the point of care assay of human neutrophil activation via elastase activity. It is easy to envision that it can be made smaller, more durable, and battery powered for use outside of a clinic, such as in rural or military applications.

Another exemplary embodiment may incorporate an assay for the number of neutrophils, separate from the total elastase activity. This could be accomplished by measuring DNA content using a known dye such as DAPI or Hoechst 33342, which fluoresces only when bound to DNA. Alternatively, cell number could be quantified from protein content, using the absorbance of the purified PMNs at an absorbance of 280 nm, or a colorimetric or fluorescent dye for protein. This would have the advantage of providing separate values for PMN number and PMN elastase activity, in addition to reporting the additive effect of number and activity per cell.

Another exemplary embodiment may capture the PMNs onto a flat glass or other transparent surface by immobilizing the anti-CD15 antibody on that surface. Once captured on the surface, an elastase substrate that penetrates viable cells (cell permeable or vital stain), could then be used to assay the elastase activity in each of the captured cells. This would provide PMN number and activity for each cell, whereby the current embodiment reports an aggregate activity of all cells. Optical imaging combined with image analysis could also determine other potentially valuable attributes of the PMNs, such as their maturity, which is reflected in their size, nuclear structure, and granularity.

Another embodiment of the technology may incorporate various types of standard wireless communications into the device so that it could report operations and results to an application on a nearby network hub, cell phone, or tablet. One can readily envision that the device would use some type of barcode reader or QR code reader to collect the specifics of the block, reagents, and the patient being tested.

Moreover, elastase is only one of several enzymes that could be measured to inform the user of the neutrophil activation state. The RNA data provided herein shows that myeloperoxidase (MPO) and alkaline phosphatase (ALPL), for instance, would likely provide useful information about PMN activation.

Furthermore, in addition to direct enzyme activity assays, such as elastase, alkaline phosphatase, or myeloperoxidase, one can envision that other indirect systems, such as antibodies to the biomarker labeled with reporter molecules, such as an enzyme or fluorochrome, could be used to measure the activation of a captured population of cells.

In addition to assaying other activation markers, one could capture and quantify other types of cells from blood as a measure of immune or autoimmune defects using the CyBIS system. For instance, one could capture specific subsets of T cells using anti-CD4 or anti-CD8 antibodies and then measure their levels of immune function reporters, such as defensins or T cell receptors.

In certain embodiments, the system uses the elastase activity to monitor the efficacy of a therapeutic treatment, such as antibiotics, over the course of time. A common problem in medicine is uncertainty over whether a particular antibiotic is effective in a given infection.

Because the elastase activity that is measured is not restricted to human subjects, the CyBIS assay would work on practically any species, including all mammals, with the only major restriction being that the anti-CD15 antibody should be reactive with the PMNs of that species. The antibody coated beads may easily be adapted to carry an antibody appropriate for any particular species.

In certain embodiments, the fluid reagents are contained in external tubes that are selectively pressurized by switchable air valves to flow the specific reagent into the reaction chamber. Other embodiments may make those fluid reservoirs integral to the disposable block and use microfluidic valves within the block to control the selection of the proper reagents.

In certain other embodiments, the PMNs are captured by paramagnetic beads coated with a monoclonal IgM directed at CD15. It is possible that a given subject may produce a form of CD15 with low reactivity to this IgM preparation, and thus, an alternate embodiment may be to utilize beads coated with multiple different antibodies to CD15, and even antibodies to other PMN markers, to produce a robust and diverse capture strategy.

Another embodiment is a method for detecting immune cell activation in a blood sample comprising:
(a) mixing antibody-coated magnetic (e.g., paramagnetic or superparamagnetic) beads with the blood sample;
(b) introducing an enzyme substrate to the contents of cells from the blood sample which are complexed with the antibody-coated magnetic beads, where the enzyme substrate cleaves in the presence of a neutrophil enzyme resulting in a product which absorbs light at a specific wavelength; and
(c) measuring light transmittance at the specific wavelength through the enzyme substrate exposed to the contents of the cells, wherein a change in the light transmittance is indicative of immune cell activation in the blood sample. In one embodiment, step (a) further comprises separating the cells in the blood sample which are complexed with the antibody-coated magnetic beads from the remainder of the blood sample by applying an electromagnetic field. For instance, the separation can be performed by washing away with a washing buffer the cells and proteins from the blood sample which are not complexed with the antibody-coated magnetic beads. In another embodiment, step (b) comprises (i) lysing the cells complexed with the antibody-coated magnetic beads to form a lysate, and (ii) exposing the enzyme substrate to the lysate. For instance, step (b) can comprise adding a buffer which (i) facilitates lysis of the cells from the blood sample which are complexed with the antibody-coated magnetic beads and (ii) contains the enzyme substrate. In yet another embodiment, step (b) comprises exposing the cells complexed with the antibody-coated magnetic beads to the enzyme substrate, where the enzyme substrate is capable of penetrating the cells. In one embodiment, step (c) comprises transmitting light through the enzyme substrate exposed to the contents of the cells and then through a bandpass filter. The light can be transmitted through a collimating lens prior to passing through the enzyme substrate which has been exposed to the contents of the cells. In one embodiment, the light is at a wavelength of approximately 405 nm. In one embodiment, step (c) comprises measuring the light transmittance at thirty second intervals. The change in light transmittance indicates enzyme activity in the blood sample. In one embodiment, the antibody-coated beads comprise anti-CD15 antibody covalently bound to the beads.

In one embodiment, the beads are paramagnetic. In a preferred embodiment, the beads are superparamagnetic. The beads may be electromagnetic.

In a preferred embodiment, the cells which complex with the antibody-coated beads are polymorphonuclear neutrophils.

In one embodiment, the enzyme substrate (such as an elastase substrate) comprises p-nitroanilide. The enzyme substrate may be an elastase substrate where the neutrophil enzyme is elastase. The enzyme substrate can alternatively be a myeloperoxidase (MPO) substrate or alkaline phosphatase (ALPL) substrate.

Yet another embodiment is a method of treating a patient comprising (a) diagnosing a patient as having an infection by the method for detecting immune cell activation in a blood sample described herein (where the blood sample is from the patient), and (b) upon identifying immune cell activation in the blood sample by observing elastase activity above preset normative values, then treating the patient for an infection.

Yet another embodiment is a method of treating a patient comprising:
(a) diagnosing a patient as having an infection by (i) mixing antibody-coated magnetic beads with a blood sample from the patient; (ii) introducing an enzyme substrate to the contents of cells from the blood sample which are complexed with the antibody-coated magnetic beads, where the enzyme substrate cleaves in the presence of a neutrophil enzyme resulting in a product which absorbs light at a specific wavelength; and (iii) measuring light transmittance at the specific wavelength through the enzyme substrate exposed to the contents of the cells, wherein a change in the light transmittance is indicative of immune cell activation in the blood sample; and
(b) upon identifying immune cell activation in the blood sample, treating the patient for an infection.

Yet another embodiment is a method of treating a patient diagnosed with an infection comprising treating the patient for an infection, wherein the patient is diagnosed as having an infection by (i) mixing antibody-coated magnetic beads with a blood sample from the patient; (ii) introducing an enzyme substrate to the contents of cells from the blood sample which are complexed with the antibody-coated magnetic beads, where the enzyme substrate cleaves in the presence of a neutrophil enzyme resulting in a product which absorbs light at a specific wavelength; and (iii) measuring light transmittance at the specific wavelength through the enzyme substrate exposed to the contents of the cells, wherein a change in the light transmittance is indicative of immune cell activation in the blood sample; and upon identifying immune cell activation in the blood sample by observing elastase activity above preset normative values, then diagnosing the patient as having an infection.

In one embodiment, the step of adding an elastase substrate comprises (i) lysing the cells complexed with the antibody coated beads to form a lysate, and (ii) mixing the elastase substrate with the lysate.

In one preferred embodiment, the antibody is an anti-CD15 antibody.

In one embodiment, the treatment step comprises administering one or more antibiotics, antibiotics, antivirals, antifungals, antiparasitics, or any combination of any of the foregoing to the patient. In another embodiment, the treatment step comprises administering one or more antibiotics to the patient.

In another embodiment, the enzyme substrate is an elastase substrate and the neutrophil enzyme is elastase.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a graph showing Droplet Digital PCR (ddPCR) quantification of specific biomarkers in whole blood RNA from patients presenting with (COVIDs) or without (Controls) COVID-19, as defined by detectable levels of the SARS-CoV2 RNA virus in nasal swabs. Bars are mean transcript level, as a percent of the ACTB RNA internal standard, ±standard error of the mean, SEM;

FIG. 2 is a graph showing relative fold changes in RNA levels, as measured by RNA sequencing (RNAseq) of differentially expressed genes (DEGs) in patients with COVID-19 syndrome that received vasopressors for symptomatic support related to sepsis;

FIG. 3 is a graph showing the relative levels of specific RNA biomarkers in whole blood from patients with or without abdominal pain;

FIG. 4 is a graph showing a kinetic assay of neutrophil elastase activity on cytocaptured PMNs from COVIDs or Controls. For demonstration purposes, 4 patients with COVID (filled circles, filled squares, filled triangles, open circles) had PMNs isolated by magnetic capture followed by a fluorometric elastase with repeated measures every 20 minutes for 1 hour. COVIDs are compared to lab controls (open diamonds, n=2) without known SARS-CoV2 infection; modified from Wargodsky et al. 2022, PloS one. 2022; 17(1):e0261679, FIG. 7.

FIG. 5A is a graph showing the number of purified CD15+ neutrophils from control (n=9) vs COVID19 patients (n=6) based on physical cell counting after the magnetic capture.

FIG. 5B is a graph showing elastase activity in purified CD15+ neutrophils from control vs COVID19 patients based on total elastase activity; Figures modified from Wargodsky et al 2022, FIG. 8.

FIG. 5C is a graph showing elastase activity in purified CD15+ neutrophils from control vs COVID19 patients based on the calculation of elastase activity per cell (elastase activity per thousand (K) captured CD15+ cells);

FIG. 6 is a graph showing elastase activity in purified CD15+ PMNs vs plasma from patients with or without COVID. Elastase activity, in relative fluorescence units (Y Axis) was measured in freeze-thaw lysates of purified CD15+ neutrophils from COVID-19 patients (n=5, solid line, filled diamonds) versus elastase activity of CD15+ cells from control patients (n=3, solid line, filled circle). Elastase activity was measured in plasma (n=5, dashed lines, open squares or circles) from the same patients with (open squares) or without (open circle) COVID-19 infection. Points reflect the mean fluorescence (Y axis) at each time point (X axis). Plasma elastase from COVID-19 and Controls are low and nearly superimposed; modified from Wargodsky et al 2022, FIG. 9.

FIG. 7A is a graph showing elastase activity and CD15+ count in isolated neutrophils versus RNA biomarker levels. A subset of patients (n=15) were divided by a positive (+, n=7) or negative (−, n=8) RNA biomarker score to identify patients with or without an abdominal infection. The average (±s.e.m.) DEFA and ALPL+IL8RB biomarker levels are shown as a percent of the ACTB RNA level measured in parallel. The asterisk * indicates p<0.05.

FIG. 7B is a graph showing elastase activity and CD15+ count in isolated neutrophils with patients grouped by the RNA score. A subset of patients (n=15) were divided by a positive (+, n=7) or negative (−, n=8) RNA score (FIG. 7A) to identify patients with or without an abdominal infection. The average neutrophil elastase activity in the CD15+ isolated cells, and the count of the CD15+ cells is shown. For graphic purposes the elastase activity is divided by 100 to put in on a scale of K/hr, similar to the count of CD15+ cells (K) per 50 ul of whole blood. The asterisk * indicates p<0.05.

FIG. 8 is a graph showing neutrophil elastase activity in patients classified by clinical risk of infection. EDTA-treated whole blood was subjected to CD15+ cell isolation with magnetic beads, and then the resulting cells were lysed, and assayed for elastase activity using a peptide substrate that liberates a fluorescent moiety when cleaved. The fluorescence in arbitrary units (AU) is thus proportional to elastase activity in the cell lysate. Patients with a diagnosis consistent with an infection (Likely Infection, solid circles, n=3) were average at each time point (1-120 min) and compared to Uncertain Infection, (solid squares, n=5), or Unlikely Infection (open triangles, n=7). The matching equations report the linear best fit to the time series data whereby the slope of the line is the velocity of the reaction (Vmax=123.94 for Unlikely Infection group).

FIG. 9 is a schematic design of the CyBIS device with CyBIS block, in accordance with an exemplary embodiment.

FIG. 10(a) is overall structural design of the air and fluid flow in the device, in accordance with an exemplary embodiment.

FIG. 10(b) is an enlarged view of the CyBIS block.

FIG. 11 is a diagram of the electrical layout of the CyBIS device, in accordance with an exemplary embodiment.

FIG. 12 is a graph showing a CyBIS POC assay on control subjects. The complete CyBIS system was applied to a normal control subject to demonstrate the automated purification of CD15+ and quantification of PMN elastase activity over time without user intervention. The Y-axis reports the CyBIS measured % Transmission of 405 nm light through the reaction chamber (filled circles) or the calculated Absorbance (filled squares) as a function of time in minutes (X axis). A linear fit equation (dashed line) is shown with the associated R², as a measure of the goodness of fit. Over the 15 minute period, the slope of absorbance (Vmax) is calculated as 0.0733 OD/min, which converts to 73.3 milliOD/min (mOD/min). In actual use, the first 5 min slope (109.4 mOD/min) is optimal because at higher activity the curve can plateau after 5 min and reduce the apparent Vmax.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

The technology described herein, referred to as “CyBIS,” utilizes circulating PMNs as highly evolved biosensors for any pathogen, allowing biothreat and infection detection days to months prior to the deployment of a pathogen-specific test. From a fingerstick of blood (about 50 ul), CyBIS rapidly captures PMNs and quantitates their neutrophil elastase activity, which reflects the number of PMNs captured, and additionally, whether the PMNs are activated. PMN elastase activity is an intrinsic part of the innate immune response. Upon immune stimulation by a variety of pathogens, elastase is liberated from intracellular granules and transported to the nucleus, where it cleaves histones to produce anti-microbial peptides, and unwinds the chromatin for export as ‘neutrophil extracellular traps’ (NETs). Thus, CyBIS is using the highly evolved functions of the PMN to detect pathogen infections anywhere in the body. Unlike circulating plasma levels of just one biomarker detected antigenically, CyBIS is using live, functioning PMNs to detect the activation of innate immunity by a variety of pathogens.

The present technology has numerous strategic and technical advantages over prior techniques. First, a major strategic advantage is that while it is known that people with infections have increased numbers of circulating PMNs, until the present studies, it was not possible to quantitate neutrophil counts at the point of care in a rapid test.

Second, while it was known that infections tend to increase circulating PMN numbers, it was not known that the elastase activity per PMN, when measured by the present techniques, is also increased. This creates an amplifying effect above simply measuring the PMN count.

Third, isolating neutrophils without compromising their integrity is an important aspect of measuring their elastase activity, and yet several published methods of isolation, such as density centrifugation, are either inadequate in yield or compromise the integrity of the ‘fragile’ PMNs. By employing magnetic beads coated with specific antibodies, a rapid (20 min), gentle isolation is achieved that is amenable to automation, as shown in the present invention. As such, pairing a paramagnetic bead isolation with enzymatic assay for elastase is novel and advantageous. Paramagnetic is used to mean that the beads, typically 1-5 microns in diameter, contain a metal such as iron, and are strongly attracted to magnetic fields, but are not magnetic themselves. Any suitable bead can be used, such as Dynabeads CD15 provided by ThermoFisher Scientific. In addition, any suitable object can be utilized, whether having a bead shape or any other suitable shape or material. In certain embodiments, the beads or objects have anti-CD15 antibodies covalently bound to the beads or objects. In further embodiments, the beads or objects are superparamagnetic, whereby specific metallic alloys are configured to amplify their susceptibility to magnetic fields.

Fourth, the neutrophil elastase assay is most valuable if it can be rendered into a format that: (1) removes user-associated variations (i.e. standardized); (2) couples cell capture directly into the enzyme assay for speed; and (3) measures enzyme activity in a kinetic assay that reports the net enzyme activity as a rate constant; and (4) performs the above functions with little to no user intervention. No such device currently exists.

Fifth, CyBIS has numerous technical advantages over preexisting technologies in biothreat defense and diagnostics/treatment, including, but not limited to: (1) the pathogen-agnostic strategy provides >100-fold faster response to evolving biothreats; (2) CyBIS requires only 50 ul of blood from a fingerstick vs ˜3 ml of blood by venipuncture for existing tests; (3) CyBIS provides results in ˜30 minutes at the point of care (POC) (4) PMN cytocapture provides ˜50-fold greater sensitivity to measure PMN activation markers because the PMNs are concentrated and the plasma inhibitors are removed; (5) CyBIS measures PMNfuncdon versus a released byproduct, which is typical of existing assays on plasma; (6) the captured PMNs can be preserved for future analysis, which would allow rapid retrospective re-analysis to detect the specific pathogen, provided it is present in the PMNs; (7) both the microfluidic block and CyBIS device can be manufactured in the United States; (8) the block can be printed by either 3D printing or by injection molding; (9) the reagents are relatively simple, inexpensive, scalable, and stable; (10) the CyBIS results are an enzyme activity rate constant that is proportional to the degree of immune activation (i.e. quantitative), not positive/negative qualitative, for the host response to infection; (11) the results are available digitally, as opposed to a colored band on a lateral flow device, for integration into an electronic health record; (12) CyBIS is readily adaptable to an in-home format, similar to a glucose monitor; and (13) CyBIS requires low power, and could be manufactured in a manner sufficiently rugged for use in rural, military, and international use.

Furthermore, the present technology overcome numerous challenges in the art. First, enzyme activity can be read by the generation of a cleaved product that absorbs light at a specific wavelength. However, generating and measuring the absorbance without generating heat and off-target fluorescence/luminescence has special issues, as described herein.

Second, the movement of small fluid volumes through small channels or tubes has special issues that cause very high shear forces that will disrupt the binding of the beads to the cells and thereby prevent capture. There is an intrinsic trade-off between efficient mixing and washing of the cells versus shear forces that can disrupt cell capture efficiency, or could fully disrupt cell integrity.

Third, the magnetic capture of the bead/cell complex should be fast and reversible to enable washing, which may be resolved by the use of an electromagnet, or movable static magnet, in proximity to the isolation chamber in the present technology.

### Definitions

Subjects: In the present examples, the subjects are healthy human control subjects, or patients that present to a physician with some type of pain or discomfort that could be caused by various types of pathogenic infections with viruses, fungi, molds, parasites, bacteria, or bacterial biofilms. Future subjects that are clearly amenable to the CyBIS approach would include other living species of the animal kingdom, such as dogs, cats, horses, and cattle.

Biofilm: Biofilms are communities of bacteria and other pathogens that have changed their metabolism and physical composition in a process involving ‘quorom-sensing’, similar to density-dependent activation, such that they aggregate, communicate, and secrete extracellular proteins that act like a shield. That protective structure, composed of proteins, proteoglycans, and lipids, can prevent the penetration of immune cells, and many antibiotics, which makes the biofilm difficult for the host, or therapeutics, to kill. In the case of appendicitis, for example, it is generally accepted that the inflammation of the appendix is caused by biofilm-forming bacteria, such as Fusobacterium nucleatum. Swidsinski A, Dorffel Y, Loening-Baucke V, Tertychnyy A, Biche-Ool S, Stonogin S, et al. Mucosal invasion by fusobacteria is a common feature of acute appendicitis in Germany, Russia, and China. Saudi J Gastroenterol. 2012; 18(1):55-8. An evolving area of therapeutics are adjuvant treatments that disrupt the biofilm, thereby allowing immune cells and antibiotics to penetrate the biofilm.

Elastase activity: Humans, and most mammals that have been examined, possess and utilize multiple forms of enzymes with elastase-like activity. In its broadest definition, elastase is any enzyme that prefers to cleave the protein elastin. The particular enzymes targeted by the CyBIS test are neutrophil elastase (a product of the ELANE gene), cathepsin G (a product of the CTSG gene), and proteinase 3 (a product of the PR3 gene), although others are known. Each enzyme has some preference for particular peptide sequences, and thus, some specificity in which enzyme is measured can be partially controlled by the particular peptide sequence used in the cleavable elastase substrate. Elastase activity is generally restricted to intracellular functions, and to pericellular functions in the secreted NET, or to facilitate cellular movements, see below. Elastase activity has been difficult to measure in plasma because it is rapidly bound by inhibitors in plasma, such as α(1) proteinase inhibitor/α1-trypsin inhibitor (α1AT), secretory leucocyte proteinase inhibitor (SLPI), and elafin. Elastase activity is a very primitive function of cells that exists is essentially all species from bacteria, where it is used by the bacteria to degrade host proteins, through most all of the animal kingdom, where it has been adapted for immune defense to allow migration of the immune cells through tissue, and in the end-stage, to degrade the pathogens themselves.

Elastase substrate(s): As described herein, the peptide sequence is bis(N-benzyloxycarbonyl-L-Ala-Ala-Ala-Ala)rhodamine (Anaspec) for the fluorescent substrate, and N-methoxysuccinyl-Ala-Ala-Pro-Val-p-nitroanilide (Sigma), where p-nitroanilide (pNA) is colorless until it is cleaved from the peptide, generating free pNA that absorbs light with a peak at 405 nM (colorimetric substrate). The substrate, and the reporting group, whether fluorescent, colorimetric, or luminescent, can be modified in a variety of ways known to anyone skilled in the art of cleavable enzyme substrates, and should be compatible with the CyBIS design, provided that the light source, filters, and sensor are adjusted appropriately.

Polymorphonuclear (PMN) lymphocytes: PMNs, also known as neutrophils, are white blood cells that are part of the innate immune system, meaning that they do not need to be ‘educated’ about what represents a threat to the organism. PMNs have the ‘innate’ or ‘natural’ ability to recognize viruses, bacteria, yeasts, fungi, and molds, and then to either engulf and degrade the pathogen, or, if that is not possible, they secrete a complex anti-pathogenic net, termed a ‘neutrophil extracellular trap’ (NET), that immobilizes and compromises the pathogen. In contrast, most lymphocytes in the T and B cell families need to be ‘educated’ about a particular pathogen by specialized ‘antigen-presenting cells’, and thus are said to be part of the ‘acquired’ immune system. In the present examples, PMNs are operationally defined as exhibiting the CD15 antigen on their surface. CD15, which has many other names, is a specialized carbohydrate structure that is added to surface proteins by the protein product of the fucosyltransferase 4 (Fut4) gene. Published studies have shown that PMNs isolated by positive selection with antibody-coated beads have better function and higher purity than PMNs purified by other methods, such as density centrifugation.

CyBIS System, Device, and Block: For clarity, the term “CyBIS” or “CyBIS system” will be used to denote the entire process of blood collection, processing on the microfluidic Block, and reading in the CyBIS Device to render PMN elastase activity at the point of care.

### EXAMPLES

The following examples help explain some of the background and operating concepts of the current invention. In particular, these examples are used to illustrate how the invention was discovered by using RNA biomarkers to point toward specific cellular changes and protein enzymes that would make suitable targets for a rapid point-of-care device. However, the general concepts of the current invention are not limited to the particular examples.

Identification of Human Host Defense Activation Biomarkers by Whole-Genome RNA Transcript Profiling of Patients Presenting with Abdominal Pain

In our published studies on 69 patients, specially preserved whole blood RNA from patients presenting with lung or abdominal infections was utilized to identify RNA biomarkers of internal infections compared to non-infectious conditions, such as a hernia ((1) Chawla et al, 2016, which is hereby incorporated by reference). Chawla L S, Toma I, Davison D, Vaziri K, Lee J, Lucas R, et al. Acute appendicitis: transcript profiling of blood identifies promising biomarkers and potential underlying processes. BMC Med Genomics 2016; 9(1):40. Some of these RNA biomarkers are disclosed in U.S. Pat. No. 11,066,706. Those biomarkers may be used as a part of the techniques disclosed herein to help define the presence of absence of infection.

The following findings inform the present technology. First, infections caused >20-fold increases in specific PMN RNAs, especially Defensin-alpha1 (DEFA1), alkaline phosphatase (ALPL), and interleukin-8 receptor beta (IL8RB, aka CXCR2). Second, in pulmonary infections, where pathogens are ‘edible’ by PMNs, the DEFA1 mRNA increased from only ˜5% of the actin beta (ACTB) control, to as much as >500% of ACTB (100×). Third, conversely, in biofilm infections, such as appendicitis, where the pathogens are encapsulated (2), DEFA1 was normal or decreased, but the levels of ALPL and IL8RB were increased >10-fold. These PMN activation transcripts (NATs) were confirmed by droplet digital RT-PCR (ddPCR).

Confirmation of Host Immune RNA Biomarkers in Patients Presenting with COVID-19 Syndrome Related to the SARS-CoV2 Viral Infection

Whole blood RNA from COVID-19 patients was analyzed by RNAseq on the Illumina platform to determine whether activation of the immune system could be detected. Differentially expressed genes (DEGs) between COVID patients and controls are shown in Table 1 (adapted from Wargodsky et al, 2022, which is hereby incorporated by reference, Wargodsky R, Dela Cruz P, LaFleur J, Yamane D, Kim J S, Benjenk I, et al, and McCaffrey, T A. RNA Sequencing in COVID-19 patients identifies neutrophil activation biomarkers as a promising diagnostic platform for infections. PloS one. 2022; 17(1):e0261679). As shown in Table 1, several of the prior abdominal pain markers, such as DEFA1, ALPL, and MPO, were confirmed in the SARS-CoV2 (likely Wuhan strain)-infected patients. For instance, RNAseq of whole blood of COVIDs (n=17) vs Controls (7) confirmed ˜20-fold induction of DEFA1 mRNA levels.

Note, in particular, that the transcript for cathepsin G (CTSG) is elevated 13-fold in persons with COVID. CTSG is one member of the neutrophil elastase group of gene transcripts. There are thought to be at least 3 members to this group: cathepsin G (CTSG), neutrophil elastase (ELANE), and proteinase 3 (PR3). This observation suggested that if the RNA for an elastase such as CTSG was activated, then elastase protein and activity may be an indicator of neutrophil activation.

TABLE 1
Differentially expressed genes (RNA transcripts) that were increased
(top) or decreased (bottom) in patients with the COVID-19 syndrome

| Fold | Control | COVID | Gene | | Cell |
|---|---|---|---|---|---|
| Change | Expression | Expression | Symbol | Transcript Description | Type |
| INCREASED | | | | | |
| IN COVIDs | | | | | |
| 3.45 | 4.76 | 6.55 | ALPL | alkaline phosphatase, liver/bone/kidney | neutrophil |
| 9.90 | 0.80 | 4.10 | BPI | Bactericidal Permeability Increasing Protein | neutrophil |
| 13.41 | 0.22 | 3.96 | CTSG | cathepsin G | neutrophil |
| 20.00 | 0.36 | 4.69 | DEFA1 | Defensin 1 | neutrophil |
| 25.80 | 2.91 | 7.60 | DEFA3 | defensin, alpha 3, neutrophil-specific | neutrophil |
| 20.53 | 1.54 | 5.90 | DEFA4 | defensin alpha 4 | neutrophil |
| 5.87 | 6.05 | 8.60 | IG*** | Immunoglobin transcripts (12 observed) | B cell |
| 8.26 | 4.10 | 7.15 | IL1R2 | interleukin 1 receptor type 2 (decoy receptor) | multiple |
| 33.34 | 0.63 | 5.69 | MMP8 | matrix metallopeptidase 8 | neutrophil |
| 7.46 | 4.40 | 7.30 | MMP9 | matrix metallopeptidase 9 | neutrophil |
| 16.12 | 0.71 | 4.72 | MPO | myeloperoxidase | neutrophil |
| DECREASED | | | | | |
| IN COVIDs | | | | | |
| −3.41 | 4.18 | 2.41 | CD28 | CD28 molecule, co-stimulatory | T cell |
| −3.91 | 6.66 | 4.70 | CD3D | CD3d molecule, T cell receptor Delta chain | T cell |
| −4.52 | 5.04 | 2.87 | CD4 | CD4 molecule | T cell |
| −2.78 | 1.97 | 0.49 | GATA3 | GATA binding protein 3 | T cell |
| −2.73 | 2.74 | 1.29 | ICOS | inducible T-cell co-stimulator (ICOS) | T cell |
| −3.43 | 2.90 | 1.13 | IKZF2 | IKAROS family zinc finger 2, aka Helios | lymphocytes |
| −5.37 | 4.85 | 2.43 | KLRB* | killer cell lectin like receptors (8 observed) | NK cells |
| −2.52 | 8.65 | 7.31 | RPS/L* | ribosomal proteins (32 observed) | multiple |
| −3.50 | 4.83 | 3.02 | TCF7 | transcription factor 7 (T-cell specific, HMG-box) | T cell |
| −3.71 | 3.46 | 1.57 | TIGIT | T cell immunoreceptor with Ig and ITIM domains | T cell |
| −3.71 | 2.55 | 0.66 | TR** | T cell receptors (67 observed) | T cell |
| −3.51 | 5.22 | 3.40 | ZAP70 | zeta chain of T cell receptor kinase 70 kDa | T cell |

### DEFA1 RNA in Blood is Elevated in COVID19 Patients

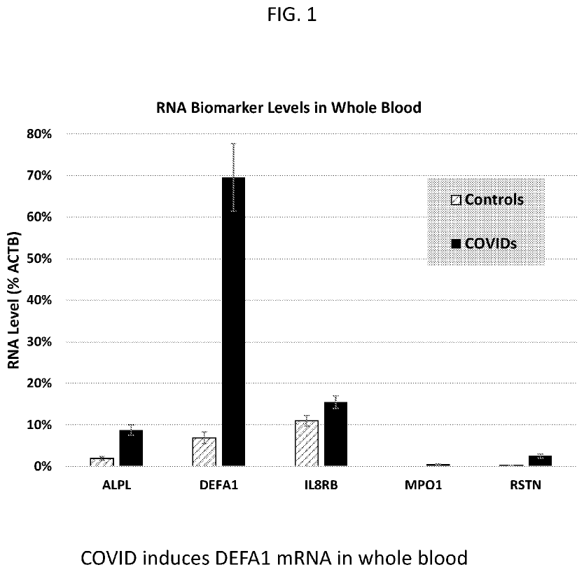

ddPCR of whole blood RNA biomarkers was confirmed in 37 COVID patients versus 12 control samples from 6 subjects, as shown in FIG. 1. The average increase in DEFA1 mRNA, expressed as a percent of ACTB transcript, was 10-fold greater than control subjects (p<0.001). This increase underestimates the true increase in the COVID patients because the COVID group includes ‘incidental’ COVIDs that were technically PCR positive for SARS-CoV2 virus, but asymptomatic. A typical DEFA1 RNA value in controls is ˜5% of ACTB, while COVID+ patients in the ICU could show ˜150% DEFA1 RNA levels (30-fold increase). The COVID-19 patients showed much smaller increases in the biofilm markers, ALPL and IL8RB, which is expected because SARS-CoV2 is not a biofilm-forming bacteria, although it cannot be excluded, and is in fact likely, that some patients develop secondary biofilm infections.

RNAseq of COVID19 Patients being Treated for Septic Shock Shows Elevated DEFA3

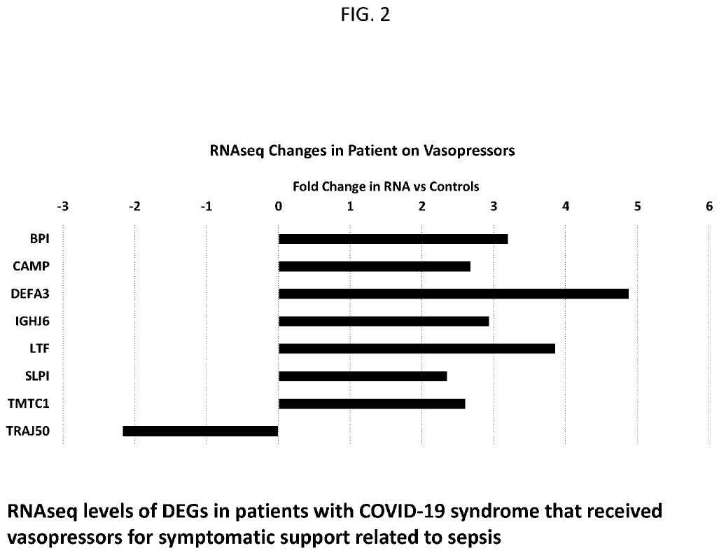

The ability of the RNA-based test, as disclosed in U.S. Pat. No. 11,066,706, and the current elastase-based method was evaluated in COVID-19 patients that minimally had SARS-CoV2 viral infection detected in nasal swabs, but also typically had other opportunistic bacterial infections. These patients had blood samples taken at The George Washington University Hospital Intensive Care Unit (ICU) and then analyzed for RNA biomarkers and elastase activity in the McCaffrey Lab. The severity of the COVID infection can be inferred from the need for vasopressor support, a sign of clinical concern for, or the actual onset of, septic shock. Septic shock is initiated by the presence of a pathogen that becomes disseminated in circulation, but is propagated by an excessive and disproportionate immune response to the pathogen, sometimes termed ‘cytokine storm’. Patients on vasopressors had almost 5-fold higher DEFA3 levels than controls not receiving vasopressors (FIG. 2). Conversely, they had lower levels of T cell related markers, such as the T cell receptor alpha joining 50 (TRAJ50) transcript (FIG. 2).

### RNA Biomarker Levels are Related to Severity of COVID Infection

Patients that were RT-PCR+ for SARS-CoV2 exhibited a broad range of clinical symptoms and severities, ranging from asymptomatic to fatal. The most severe and life-threatening conditions include septic shock, which can be empirically categorized posthoc, by the use of vasopressors to maintain blood pressure. Dividing the patients by vasopressor use indicates that patients on vasopressors had 2-fold higher DEFA1 mRNA in blood, suggesting DEFA1 may be a useful biomarker of COVID severity (data not shown).

RNA Biomarkers are Elevated in Patients Presenting with Abdominal Pain in Emergency Room (ER)

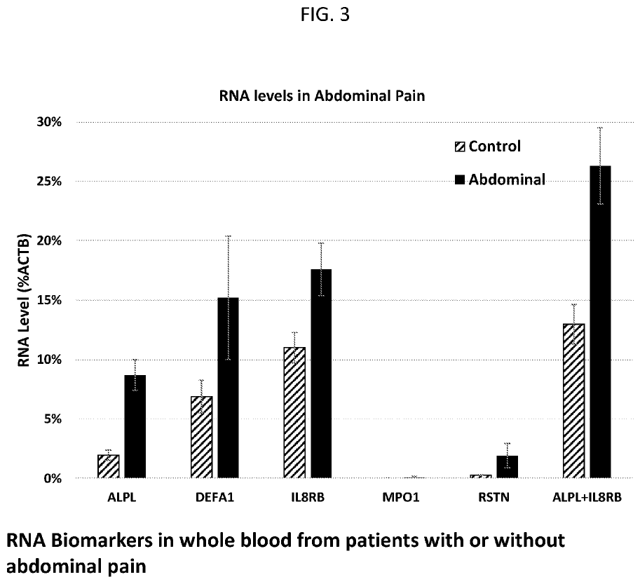

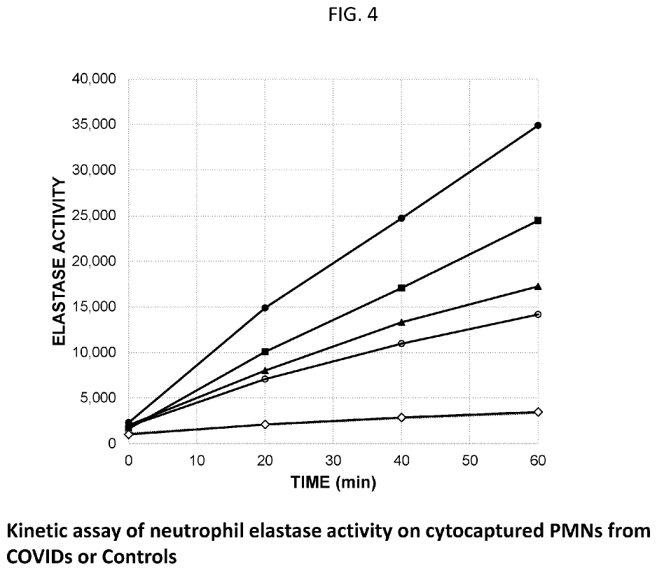

Patients presenting at the ER with clinical signs of abdominal infection, such as appendicitis, were analysed for their levels of the RNA biomarkers. As shown in FIG. 3, the levels of the main RNA biomarkers, ALPL, DEFA1, and IL8RB were elevated from 2-4-fold in patients with suspected internal infections. This initial analysis underestimates the true magnitude of the increase because many of the cases of abdominal pain were ultimately determined to be of non-infectious origin. When the patients are separated by infectious vs non-infectious causes of the pain, the infectious patients had TruNAV RNA biomarker levels that were significantly elevated over their contemporaneous ER patients with non-infectious causes of their pain. In this analysis, infectious causes were typically acute appendicitis, diverticulitis, or certain types of urinary tract infections, while non-infectious causes were typically gas, acid reflux, or sterile inflammatory syndromes such irritable bowel syndrome (IBS) or Crohn's disease.

### Analysis of RNA Biomarkers on Purified Human PMNs Via CyBIS

Immune activation is typically analyzed by: (1) density centrifugation of blood at high gravitational (g) force to isolate particular cell types; (2) immunostaining of the isolated cells, involving repeated centrifugation to wash unbound ligands; and (3) flow cytometry with laser excitation. While common, it is slow, requires large instruments, requires considerable expertise, specialized instruments, and damages fragile PMNs. Further, existing centrifuge-based methods are typically not amenable to automation. CyBIS has been refined so that it allows rapid isolation, washing, and biomarker quantitation, without the need for centrifuges or flow cytometers, by using paramagnetic beads coated with antibodies to the CD15 surface antigen on PMNs and certain eosinophils.

Using magnetic beads coated with anti-CD15 antibodies, our studies confirmed using the present technology that CD15+ PMNs have significant levels of the RNA biomarkers. Purified CD15+ PMNs, buffy coat (total white cells), and whole blood RNA from a subset of COVID19 patients (n=23) were analyzed for their levels of the RNA biomarkers by ddPCR. A correlational analysis indicated that DEFA1 RNA was poorly correlated between the CD15+ subset of purified cells and whole blood RNA (Pearson R=0.015), but the RNA levels of ALPL was well correlated between CD15-purified cell RNA and whole blood RNA (R=0.53), confirming that purified CD15+ cells are at least one source of the RNA biomarkers in blood Wargodsky et al 2022, which is hereby incorporated by reference).

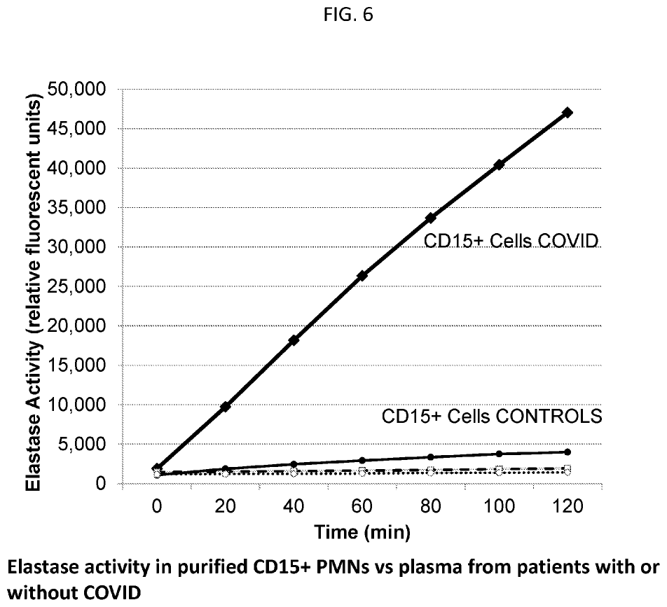

Our RNAseq analysis also revealed that PMN elastase RNAs were induced >10-fold by COVID infection (24 patients), as shown in Table 1, CTSG, Cathepsin G (modified from FIG. 1, Wargodsky et al 2022). Cathepsin G is a well characterized neutrophil elastase that has both intracellular and extracellular actions in the course of innate immunity. CD15-purified PMNs, when examined by light and fluorescent microscopy, showed a striking increase in both number and the brightness of DAPI-stained nuclei in COVIDs, suggesting active decompaction of chromatin due to histone cleavage by elastases (10 patients) (see FIG. 6, Wargodsky et al 2022).

### Adaptation of RNA Biomarkers to a Functional Assay of PMN Elastase

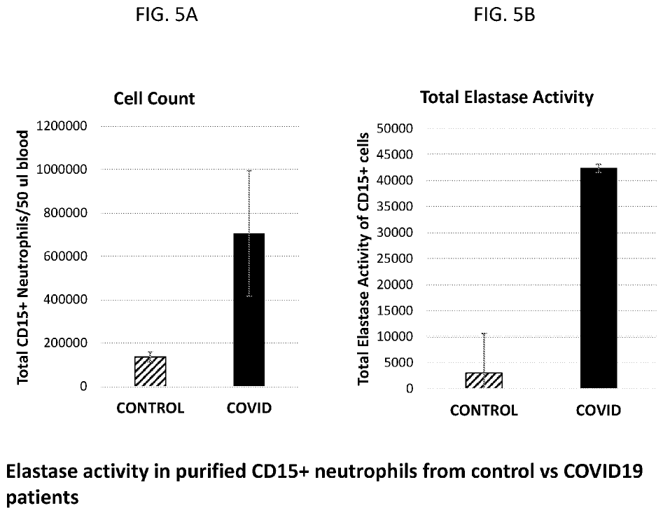

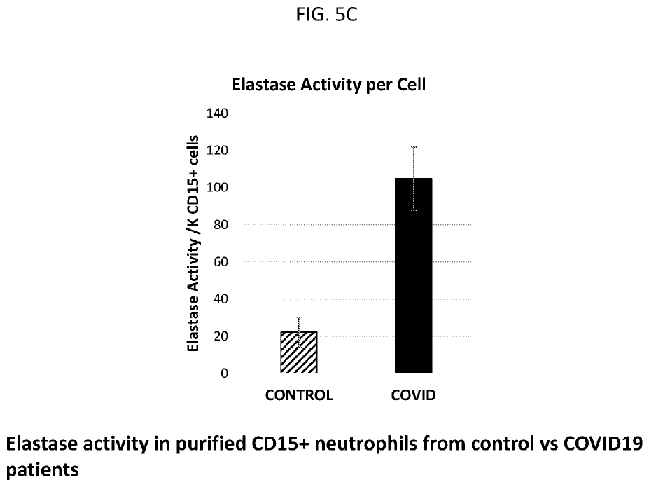

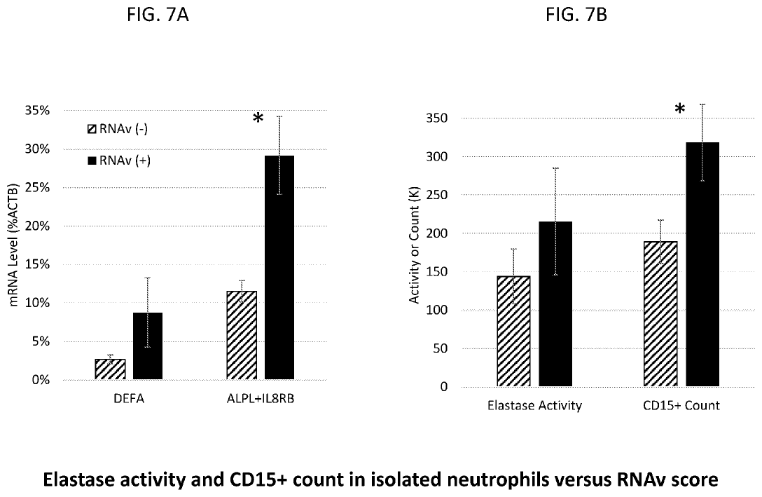

While RNA is a highly specific marker of cellular functions, it has been difficult to convert it to a POC platform for several reasons. However, these RNA targets that we identified, pointed us toward targets that are more amenable to measurement in a point-of-care setting. A highly correlated, but more simple and direct method was identified, as outlined here:
Our observation that the captured PMNs from infected patients were ‘DAPI bright’ (see FIG. 6 of Wargodsky et al 2022) enabled a key insight that cellular elastase activity, which ‘unpacks’ the chromatin and makes it accessible to the DAPI stain, hence brighter, may be a functional measure of PMN activation.
In fact, PMN elastase activity measured in a kinetic assay over a 1 hour period was up to 7-fold higher in COVID CD15+ PMNs captured from blood vs Controls (FIG. 5B).
Further, if PMNs from COVID patients were allowed to adhere to a glass slide, and then visualized with a fluorescent DNA stain (Hoechst 33342), it was apparent that they were actively spreading and releasing NETS, confirming that the captured cells are functionally active (see FIG. 7 of Wargodsky et al. 2022).

### Elastase Activity in COVID-19 Patients

A one-tube, fluorescent or colorimetric assay that captures PMNs, washes, and conducts a kinetic assay of PMN elastase activity was developed. By accurate cell counting, and fluorometric assay of elastase activity over time, it is possible to know the neutrophil count, total elastase activity, and elastase activity per cell. As shown in FIG. 5A, the number of captured CD15+ PMNs is increased from under 200K/50 ul blood to over 600K/50 ul blood. The total elastase activity increases approximately 10-fold (FIG. 5B), and the elastase activity corrected for the cell count indicates that elastase activity per PMN cell is increased 5-fold (FIG. 5C). Thus, both the number of PMNs, and the elastase activity per PMN is increased in COVID-19 patients.

In contrast, plasma elastase activity from the same patients was only 1% of the level detected in the purified cells, and there was no significant difference between the plasma elastase activity of COVIDs and Controls (FIG. 6, dashed lines, open symbols). This result is consistent with data that elastase is not secreted until a very late stage of PMN activation (NET formation), and that elastases are rapidly inactivated in plasma. This likely explains why plasma or serum elastase has not been successfully developed as a biomarker for infections.

Elastase Activity is Elevated in Patients with Abdominal Pain of Potentially Infectious Etiology

### Methods

CD15+ cell isolation: Blood from cubital fossa venipuncture was collected in 6 mL K2 EDTA Vacutainer tubes (BD Biosciences). Within 20 minutes post-collection, the CD15+ cells (comprising neutrophils, eosinophils, and a rare subtype of T cells) were isolated using anti-human CD15-coated Dynabeads (Invitrogen) and a modified version of the product's protocol. The bead isolation buffer (BIB) was modified by using HBSS without calcium or magnesium (1×; Gibco). Instead of the recommended initial wash steps, we centrifuged the blood at 3000 rpm (604 g) at 4° C. for 10 minutes, removed the plasma, and added 750 uL of BIB just before adding the beads. Cell counts of the 1× isolate were performed using 10 uL in a hemocytometer at 30× under phase. The identities of the isolated cells were confirmed histologically using a May-Grunwald stain.

Whole blood WBC, differential count, and CD15+ yield calculation: White blood cell (WBC) counts and differential counts were conducted on the same blood used for the isolation of the CD15+ cells. WBC counts were achieved by adding 20 uL of whole blood to a Leukotic tube (Bioanalytic), following the product's protocol, and counting the preserved cells in a hemocytometer. Differential staining of blood smears was achieved by fixation in methanol (10 min; ThermoFisher Scientific), eosin Y staining (40 s; Sigma Aldrich), Wright Giemsa staining (100% 1:30 min, then 50%3 min; VWR), and finishing with Permount (Electron Microscopy Sciences) and a coverslip. For each sample, 200 or more cells were counted within the feathered edges of the smears. Yield calculations were performed by comparing the isolate's cell count to the WBC's fraction of neutrophils and eosinophils as determined by the differential count.

Buffy coat (mononuclear cell) isolation: Blood from cubital fossa venipuncture was collected in 4 mL sodium citrate mononuclear cell preparation vacuum tubes (CPT; BD Biosciences) at the same time as the EDTA tubes for CD15+ cell isolation. The tubes were centrifuged in accordance with BD's guidelines (room temperature, 1500×g, 25 min). The plasma above the gel was discarded except for the last 500 uL, which was pipetted to wash the buffy coat off the surface of the gel, and transferred to a new tube. The buffy coat's mononuclear cells (lymphocytes, few monocytes and scant low-density neutrophils) were observed in a hemocytometer under phase to check the isolation process. The cells were pelleted by centrifugation (151 g, 10 min, 4° C.).

Fluorometric elastase assay: CD15+ cells for the fluorometric elastase assay were isolated by the standard isolation protocol outlined above. Aliquots of cells (50 uL) were stored at −80° C. Samples were thawed and underwent 5 cycles of freezing and thawing (dry ice slush with isopropanol; then 37° C. water bath). The substrate, bis(N-benzyloxycarbonyl-L-tetra-alanyl)rhodamine (Anaspec) was dissolved in DMSO and PBS without calcium and magnesium (DMSO:PBS=4.5:1) for a stock concentration of 2 mM. Cleavage of the substrate's two Ala-Ala-Ala-Ala (tetra-alanyl) groups resulted in the measured 520 nm (green) emission. Substrate cleavage in the lysate was initiated with 140 uL of PBS without calcium or magnesium and 10 uL of stock elastase substrate was added, the beads were pelleted 6s on a strong neodymium magnet, and the fluorescent product was measured on a Qubit 2.0 fluorometer (ThermoFisher) with 470 nm excitation/520 emission. Mixing and measurements were repeated every 20 minutes for 2 hours.

Colorimetric elastase assay: The CD15+ cells were isolated, stored, and lysed via freezing and thawing in the same way as for the fluorometric elastase assay described above. The substrate, methoxysuccinyl-ala-ala-pro-val-pam-nitroanilide (Sigma Aldrich), was dissolved in DMSO and PBS without calcium or magnesium (4.5:1) for a stock concentration of 6 mM. 140 uL of PBS without calcium and magnesium was added to each tube. The cells remnants and beads were mixed by vigorous pipetting, placed on the strong neodymium magnet for 15 s to pellet the beads, and the supernatant with the cell fragments was transferred to a clear 96-well plate. 10 uL of stock substrate was added to each and they were mixed by pipetting 10s. The VersaMax tunable plate spectrophotometer (Molecular Devices) was used to read the 410 nm absorption of the cleaved substrate in each well every 5 minutes for two hours. In this example, the Vmax (milli OD/min) was calculated based on the first 15 minutes.

### Results

Functional activation of isolated neutrophils. From the above-mentioned work in COVID-19 patients admitted to the intensive care unit (ICU), it was observed that elevated RNA scores were frequently associated with increased CD15+ neutrophil counts, and with elevated neutrophil elastase activity in the magnetically isolated cells. To determine whether that held true in abdominal pain patients, CD15+ neutrophils were magnetically isolated, washed, and then subjected to nuclear staining with Hoechst stain (or DAPI) to image and count the nuclei using automated image analysis. Patients with elevated biofilm scores (ALPL+IL8RB) also exhibited elevated CD15+ cell counts, relative to similarly treated patients with apparently non-infectious bowel obstruction (data not shown).

Rapid kinetic assay of elastase activity. Using magnetically isolated PMNs (CD15+), NE activity was assessed with a fluorgenic substrate and it was observed that: 1) predictably, the yield of CD15+ cells was greater in patients with an intra-abdominal infection (IAI), and 2) the total NE activity was likewise elevated. As shown in FIG. 7, when IAIs were classified on the basis of the RNA score (FIG. 7A), the DEFA1 and ALPL+IL8RB RNA levels, are, of course, higher in the RNA-positive biomarker group. NE activity (FIG. 7B) was higher in RNA (+) patients (20,943, n=6) than RNA (−) patients (14,049, n=9)(graphically shown as 209 vs 140), although the difference was not statistically significant in this small sample group (p=0.35). The yield of CD15+ cells was about 1.6-fold greater in RNA (+) than RNA (−) patients (FIG. 7B, p<0.05). The statistical analysis of the elastase activity was complicated by one patient that was confirmed as RNA (−), but had strong NE activity, suggesting a potentially non-infectious cause of elevated NE activity. There is a documented relationship between gall bladder surgery and NE activity in plasma, where it is suspected that bile acids can directly activate NE release from PMNs, and this would be consistent with the final diagnosis of this patient as ‘cholelithiasis’ (gall stones).

Overall, there is strong positive correlation (Pearson R=0.652) between the elastase scores and the RNA scores, and this becomes stronger (R=0.780) when the cholelithiasis case is omitted. Further, the comparison of RNA+ vs RNA− is imperfect because while the 9 patients are technically showing low RNA levels of the biomarkers, they are presenting to an emergency room for abdominal pain. All but 2 of the 9 patients had RNA scores above 10, where 5 is a known normal level. Thus, it would be unwise to consider them ‘uninfected’ controls, when ‘potentially infected’ ED patients would be more accurate. More extensive studies of control subjects will be required to establish baseline ‘uninfected’ levels for elastase activity.

### Elastase Activity by Infection Risk Group

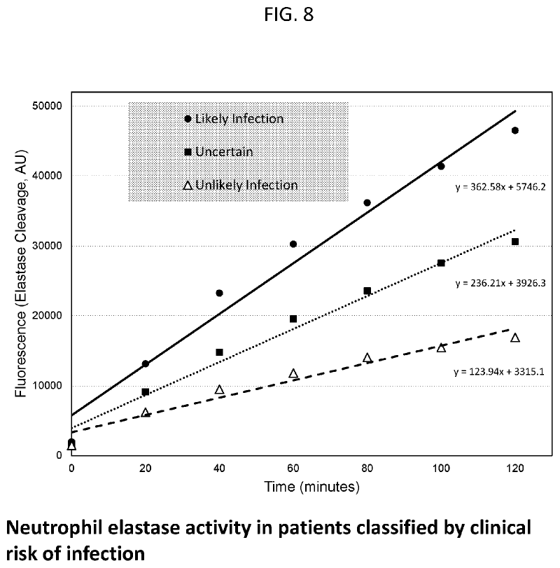

If the patients are reclassified into Infection risk based on the clinical diagnosis alone, a similar pattern emerges. As shown in FIG. 8, the risk groups show stepwise increases in NE activity over a 2 hour period from Unlikely Infection (Vmax=123.9), Uncertain Infection (Vmax=236.2), to Likely Infection Risk (Vmax=362.6).

Fabrication of CyBIS cytocapture block and reader. Using 3D printing, small polymer blocks were manufactured that contain a fluid chamber and small tubes to capture cells from anticoagulated blood in 20 minutes. These cells are then assayed for PMN elastase activity and PMN count in the same chamber in 10-15 min. The CyBIS block is ˜3×3×1 cm (l, w, d), with 2 microfluidic access ports into the 100 ul volume chamber, and a clear light path to allow quantification of light transmittance. The instrument uses a programmable microcontroller to automate magnetic capture of PMNs, washing, and the measurement of elastase activity in the captured cells via colorimetric substrate cleavage overtime.

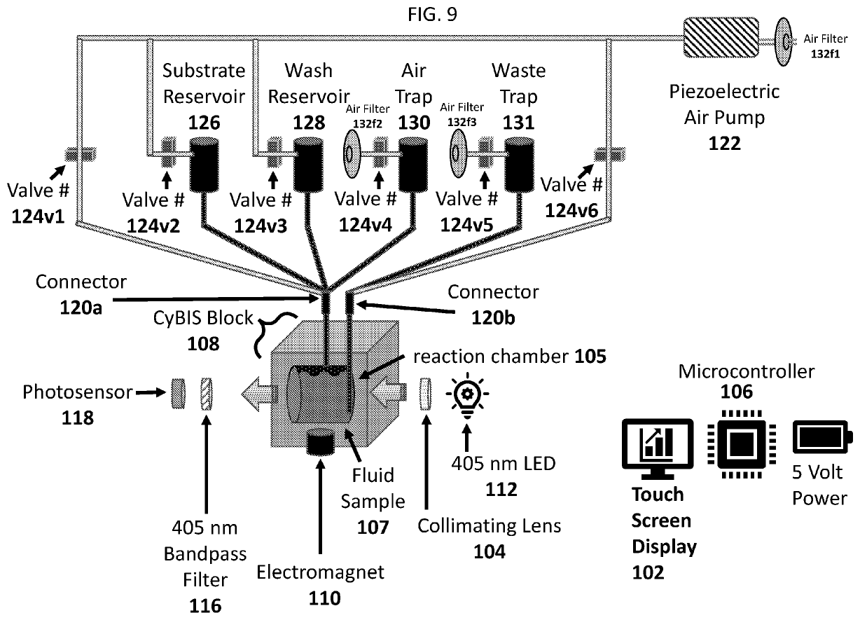

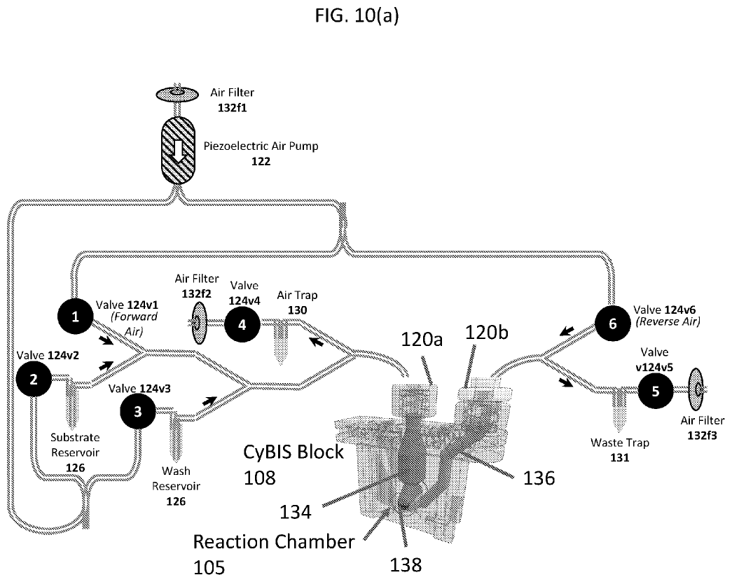

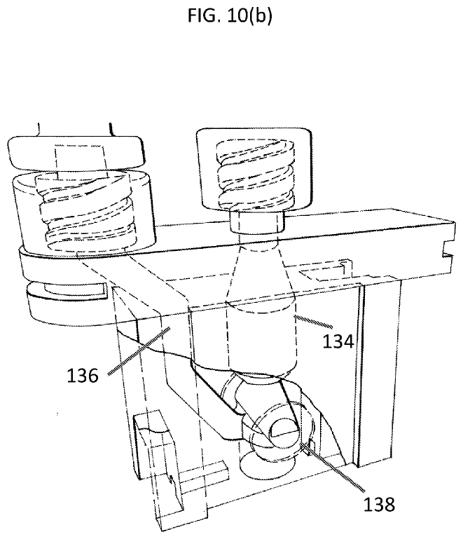

As shown in FIG. 9, the system includes a CyBIS block (consumable) 108 which is a housing for a reaction chamber with clear light path 105, one or more electromagnets 110, light source 112, collimating lens 104, bandpass filter 116, and photosensor 118. The one or more electromagnets 110 are situated adjacent to the reaction chamber 105 of the block 108. The electromagnets 110 attract the magnetic capture beads that bind to the PMNs forming a bead/cell complex. In practice, custom-made electromagnets generated unacceptable heat when powered to capture beads at ostensibly reasonable distances of ˜1 cm. As such, the present technology adapts a large electromagnet 110 in sufficient proximity (<1 cm) to the chamber 105 to bind the bead/cell unit while washing under sufficient flow rates. This electromagnet can also be replaced by a static magnet placed on a servo motor to move the magnet near to the chamber to capture cells, and then moved away to release them. The 3D design of the block 108 is shown in greater detail in FIGS. 10(a) and (b), with the fluid paths to reaction chamber 105.

A light source 112, preferably an LED source with an emission wavelength of approximately 405 nm, transmits light through a collimating lens 104 and through the reaction chamber 105, containing captured cells and the elastase substrate, which composes the sample 107. However, when the LED source 112 produces sufficient light energy to penetrate the sample 107, it can also evaporate up to 50% of the volume of the sample 107 in the chamber 105 in approximately 20 minutes, resulting in: a) increased cleavage rates, b) possible inactivation of the elastase, and c) a decrease in the volume of sample 107, which affects the transmittance of light in an unpredictable manner.

As a result, the light intensity and distance from the LED source 112 to the chamber 105 should be carefully tuned to balance between sufficient light for the photometer versus excessive heat. Also, to minimize heating, the LED source 112 is switched on for a short period before the reading, to allow it to come to a stable emission, the reading is taken, and the LED source 112 is switched off between readings (pulsed or ‘flash’ light source). A collimating lens 104 is thus used to focus the light on the reaction chamber from this greater distance. That is, the lens 104 allows the light 112 to be positioned a greater distance from the chamber 105, so that the LED 112 doesn't overheat the sample in the reaction chamber 105, while at the same time the light intensity at the photosensor 118 is maintained. An acceptable balance of light vs heat is achieved when the LED source 112 is 3 Watts (W), powered at 3.5-4 Volts (V), at a distance of about 1 cm from the reaction chamber 105 containing sample 107. In this design, the LED light source 112 will produce a full range high signal (16 bits saturated=65,536) on the photosensor 118 in the absence of any sample 107, and a roughly half-maximal signal (32,000) when a sample 107 without cleaved substrate is inserted in the light path of chamber 105. The rate of reduction in light transmittance due to substrate cleavage is directly proportional to the content of elastase in the sample, and varies from 100 milliOD units/min to 500 milliOD units/min, depending on the number and activation of the PMNs (as shown in FIG. 8).

The light from the light source 112 passes through the reaction chamber 105, containing the sample 107, and through a bandpass filter 116, which preferably has a wavelength of approximately 405 nm. A photosensor 118 produces a digital readout from the collected light that passes through the reaction chamber 105 and bandpass filter 116. Transmission of relatively pure 405-410 nm light (violet, sometimes called ‘black light’) from an LED source 112 produces a broad range autofluorescence of the cellular lysate solution, due to interaction with a range of biomolecules in the lysate. This is apparent as the glow that is observed when white clothes are seen under ‘black light’. This creates an increasing light level overtime to the photosensor 118 that offsets the absorbance of light created by cleavage of the substrate in the substrate reservoir 126 in the reaction chamber 105. As such, the 405 nm bandpass filter 116 is needed between the reaction chamber 105 and the photosensor 118 to block this spurious signal.

The CyBIS block 108 has one or more connectors 120, such as tubes or pipes that can transport air or fluid. A first inlet connector 120a connects the block 108 to various inlet air or fluid components (e.g., air from the air trap 130, wash buffer in the wash buffer reservoir 128, substrate buffer in the substrate reservoir 126) that are used in the assay. A second outlet connector 120b connects the block 108 to various outlet air or fluid components, such as fluid waste to the waste trap 131, that are expelled from the chamber 105. The air trap 130 and the waste trap 131 each have an air outlet valve 124v4 and a waste outlet valve 124v5, that lead to an outlet air filter 132f2 and a waste outlet air filter 132f3, respectively.

The connectors 120 allow the fluids used for washing 128, or elastase activity measurement 126, to flow into the reaction chamber 105, where cells are captured by the magnetic beads. An air pump 122 is used to displace fluid components out of the fluid reservoirs and into the block 108. In principle, the fluids could be moved directly by a pump, but the fluids contain salts that tend to foul pumps over time, and so positive displacement of air into a fluid chamber has the effect of isolating the pump from the fluids. Relay-switched valves 124v1-124v6 are used to direct the positive air pressure of the pump 122.

For example, to move wash buffer from the wash reservoir 128, all of the valves 124 are closed except for the wash reservoir valve 124v3 and the waste trap air vent valve 124v5. When the pump 122 is turned on, positive air pressure into the wash reservoir 128 displaces liquid into the reaction chamber 105 via the tubes and a first fluid connector 120a. Air escapes the chamber 105 through the second fluid connector 120b, via the other tubes and through the waste trap air vent valve 124v5, leaving any fluid in the waste trap 131, with displaced air escaping via the waste trap air filter 132f3. The coordination of the pump 122 and valves 124 is achieved by the microcontroller 106 which is executing a custom software program that directs the fluid flow to achieve the basic steps of washing the cells and adding substrate buffer for elastase measurement.

When the blood sample is mixed with magnetic beads coated with antibody to CD15, the mixture then composes fluid sample 107 which is placed in the reaction chamber 105 via a needle or pipet inserted through one of the connectors 120, in a volume of about 100-150 ul. As explained in greater detail below, the basic process calls for the sample 107 in the reaction chamber 105 to be gently mixed by pulsatile positive air pressure through the connectors 120 from the air pump 122. After approximately 20 minutes, the electromagnet 110 is powered, to attract the magnetic beads to the wall of the reaction chamber 105, in about 1 minute. The reaction chamber can be made of almost any material that retains liquids, provided that it does not block the magnetic field induced by magnet 110. The antibody-coated beads could be created in any of a variety of ways that would allow their physical capture, or retention, in the reaction chamber. In the present embodiment, the beads are a composite of polystyrene and iron particles with a diameter of about 4.5 microns. The beads are not themselves magnetic, but the iron is attracted to a magnetic field, and thus the beads are said to be paramagnetic.

Next, the blood/buffer sample 107 is aspirated from the chamber 105 into the waste 130, and wash buffer in the wash reservoir 128 is flowed into the chamber 105 by positive pressure in the wash reservoir 128 created by opening the wash valve 124v3 and the air pressure from the pump 122. The wash step has the effect of retaining the PMNs bound to magnetic beads, but removing irrelevant and undesirable cells, such as RBC, and inhibitory proteins, such as the elastase inhibitors in plasma. There are 3 cycles of this wash and aspiration, and then the substrate in the substrate buffer reservoir 126 is introduced by similar positive displacement created by air pressure from the pump 122, through the opened substrate valve 124v2 into the substrate reservoir 126. By adding the elastase substrate in a buffer containing a mild detergent, for example Triton X-100, the cells are disrupted and their protein contents are exposed to the substrate. Thus, the neutrophil elastase is now able to access and cleave the substrate from its colorless state to its pale yellow form that absorbs light of 405 nm in wavelength.

An overall structural design of the air and fluid flow in the device is shown in FIGS. 10(a) and (b). This aspect of the system functions to supply substrate buffer and washing buffer to the block 108, while removing waste. In practice, peristaltic pumps, combined with the fluid tension and bubble generation within narrow bore tubes created unpredictable back pressures that made dependable fluid delivery difficult. As such, the control of the small volumes required specialized piezoelectric pumps combined with positive displacement chambers to isolate the pump 122, which prefers air, from the blood and liquids needed for binding, isolation, and washing. As shown, the air pump 122 is responsible for directing air through the valve 124 that controls the substrate pump 126, the valve 124′ that controls the washing buffer pump 128, and the reverse valve 124″ that is used to draw away waste using the waste pump 130. In FIG. 9, the tubes all join once at the connectors, which is a graphic simplification, while in FIGS. 10(a) and (b), the tubes are more accurately drawn to show that they are typically joined by sequential Y-shaped connectors, but having the same general effect as in FIG. 9. Accordingly, any suitable configuration can be provided.

### CyBis Device and Block Design

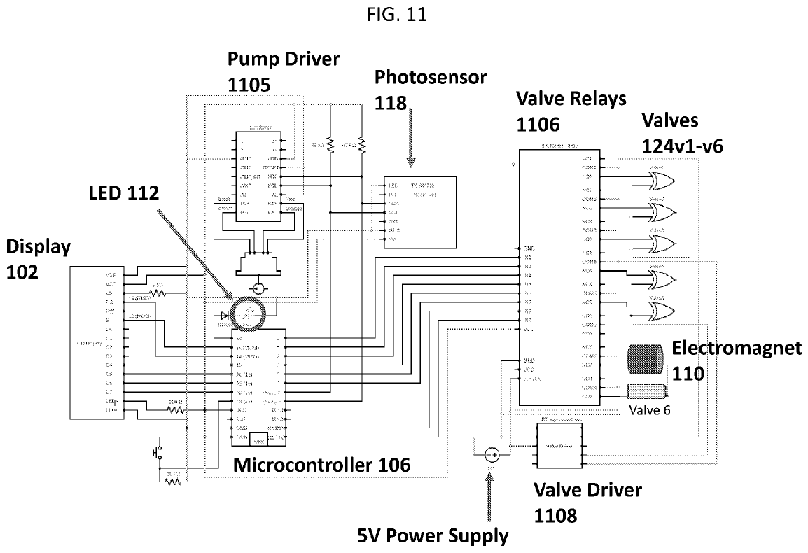

A schematic drawing of the electrical components of the device are shown in FIG. 11. At the heart of the CyBIS design is the CyBIS block 108 which would be positioned between the LED 112 and the photosensor 118. FIG. 11 shows a typical wiring diagram illustrating how the microcontroller 106 would be connected to the LED 112, photosensor 118, pump driver 1105, valve relays 1106, which control valve 124v1-v6, via the valve driver 1108, electromagnet 110, with the operations and results displayed on the display 102. Operationally, human (or other species) blood (50-75 ul), with CD15-antibody coated magnetic beads in an EDTA-containing buffer (75 ul) are manually added to begin the assay process. The mixture of blood, with an anticoagulant buffer and the magnetic beads bearing anti-CD15 antibodies collectively compose the initial sample 107 which is placed into the reaction chamber 105 of the block 108. The block 108 contains the reaction chamber 105 for all of the major steps in the reaction: bead-to-PMN binding, magnetic collection, washing, and elastase assay. The block 108 is currently 3D printed using a high-resolution UV polymerizing (stereolithography, SLA) printer (Formlabs).

In one embodiment, the block 108 is a solid structure that forms a housing. The chamber 105 is a hollowed portion formed in the block, such as by 3D printing, drilling, extrusion, or molding. FIG. 9 contains a side-view of the block 108 and chamber 105, whereas FIGS. 10(a) and (b) are perspective front views. As further illustrated in FIGS. 10(a) and (b), the block 108 is formed with an inlet channel 134 and an outlet channel 136. The inlet channel 134 extends from the top surface of the block 108 vertically downward to a bottom section of the block 108 and tapers outward to the reaction chamber 105. The inlet connector 120a is formed as a female adapter that projects upward from the top surface of the block 108. The inlet connector 120a couples with a male adapter at the end of the inlet tube leading to the reservoirs 126, 128. When the tube male adapter is coupled to the female inlet connector 120a, a liquid and airtight seal is formed to provide a sealed fluid communication between the inlet tube and the inlet channel 134. As illustrated, the connectors 120 can be internally threaded to threadably mate with an external thread on the respective inlet and outlet adapters.

In addition, the outlet channel 136 extends to the side of the inlet channel 134, upward and slightly outward from the reaction chamber 105 at the bottom of the block 108 to the top surface of the block 108. The outlet connector 120b is formed as a male adapter that projects upward from the top surface of the block 108. The outlet connector 120b couples with a female adapter at the end of the outlet tube leading to the waste trap 131. When the tube female adapter is coupled to the male outlet connector 120b, a liquid and airtight seal is formed to provide a sealed fluid communication between the outlet tube and the outlet channel 136.

Accordingly, the inlet channel 134 leads into the top of the reaction chamber 105, and the outlet chamber 136 leads out from the bottom and side of the reaction chamber 105. Accordingly, air and fluid can enter from the inlet tube, through the inlet connector 120a, into the inlet channel 134 to the reaction chamber 105. Air and fluid can exit from the reaction chamber 105, through the outlet channel 136, through the outlet connector 120b and into the outlet tube. The reaction chamber 105 is a closed chamber in air and fluid flow communication with the inlet channel 134 and the outlet channel 136. In addition, an opening 138 is formed on both sides of the block 105 adjacent to the reaction chamber 105. A glass pane is located on both sides of the opening 138, which is aligned with the light source, LED 112. Accordingly, light generated by the LED 112 enters the reaction chamber 105 through the glass panes on both sides of opening 138, to reach the sample located inside the reaction chamber 105.

The block 108 is inserted into the CyBIS Device (a box comprising the elements of FIG. 9, FIG. 10, and FIG. 11), between the collimating lens 104 and the bandpass filer 116. The CyBIS device is comprised of a plurality of control elements. The Device includes a microcontroller 106 (for instance, Arduino or Pi) with an output to a touchscreen display 102 (FIG. 9), and inputs from a photosensor 118, that is used to measure and quantify light transmitted by the LED 112 through the reaction chamber 105. The microcontroller 106 is in communication with, and controls the operation of, the display 102, LED 112, pump driver 1105, photosensor 118, valve relay controller 1106, and valve driver 1108. The relay controller 1106 controls operation of the relay-switched valves 124v1-v6 that are used to direct substrate 126 and washing buffer 128 into the block 108, while removing waste 130. The relay controller 1106 also preferably controls the one or more electromagnets 110, and is in communication with the valve driver 1108, which opens and closes the relay-switched valves 124v1-v6, as required by the system.

In certain embodiments, the block 108 is approximately 3×3×1 (h,w,d) cm in dimension with a small light path through it near the bottom. That light path is designed to be 1 cm in length, 3 mm in diameter, and about 100 microliters in volume to contain the captured cells and run the elastase assay. Preferably, the block 108 is 3D printed by stereolithography (SLA) using resins that rapidly polymerize upon UV laser activation. The reaction chamber is printed with openings at each end, and then the openings are later covered with transparent borosilicate glass coverslips, or plastic coverslips, by bonding with UV-activated resin. The block preferably has 1 input port and 1 output port that access the reaction chamber, each is fitted with a threaded twist fitting to secure the tubing. One is male and one is female so that only the correct connection can be made. The CyBIS blocks 108 need not be sterile or pyrogen-free, because they never contact the subject, only the blood from the subject.

### Steps in the CyBIS Assay

The steps in a typical CyBIS assay are outlined below. The assay begins by collecting a suitable sample, which is preferably, but not limited to, a small sample of blood collected by any of a variety of means known to anyone skilled in the art. Typically, a small lancet stick to the finger is sufficient to produce 50 ul (about 1 drop) of blood (Step 1), which is collected into any variety of containers, such as a hematocrit tube (Step 2). The blood is transferred to a tube, where it is mixed with the BIBS buffer, and the magnetic beads coated with antibody to CD15 (Step 3). The sample mixture 107 is injected into the reaction chamber 105 of the CyBIS Block 108 via one of the two connectors 120 (Step 4). The connectors 120 are secured to the block (Step 5), and the software program is initiated (Step 6).

The microcontroller 106 electrically activates the air pump 122 and valves 124v6 (reverse air) and 124v4 (vent) to create a slightly reverse fluid movement in the reaction chamber 105 to mix the beads with blood (Step 7). After a few seconds, valves 124v4 and 124v6 are closed and valves 124v1 and 124v5 are opened, which has the effect of directing the pressure in the opposite direction, thereby moving the sample fluid in the reaction forward and back, creating a gentle mixing effect. This mixing is necessary to keep the magnetic beads, which are slightly heavier than cells or protein, from settling to the bottom of chamber. Thus, the antibody-coated beads can directly the cells of interest in blood, and allow the antibody to bind to the surface marker, in this example, CD15.

After about 20 minutes, the beads, which are now bound to cells, are captured against the wall of the chamber by the microcontroller 106 activating the electromagnet 110 (Step 8). For example, the microcontroller 106 is in communication (wired or wireless) with the electromagnet 110 and transmits a control signal to the electromagnet 110 that turns ON the electromagnet 110. The blood and buffer, with unwanted cells and proteins, are then displaced from the chamber 105 by positive pressure of air, thus moving them to the waste reservoir 131 (Step 9). This is achieved by using forward air pressure from the pump directed by opening valve 124v1 and escape valve 124v5, with all other valves closed (the valves default to the closed position when not powered by the current from the valve driver).

Fresh wash buffer is then moved into the chamber by the microcontroller 106 and valve relays 1106 opening the buffer valve 124v3, closing all other valves except the waste valve 124v5, and then powering the air pump 122. With clean buffer in the reaction chamber, the microcontroller 106 turns OFF the magnet 110 to allow the beads/cells to be washed by the buffer, releasing any unwanted cells that might have been trapped accidentally by the beads (Step 10). The buffer is mixed by alternating pressures, as described above, and then the cycle of magnetic capture and washing is repeated 2 more times (Steps 11 and 12). After the last wash buffer is removed, the substrate buffer is moved into the reaction chamber, and it has a mild detergent, for instance Triton X-100, that lyses the cells, and the colorimetric substrate for elastase (Step 13).

After the substrate/detergent buffer is added, the cells are lysed, exposing their contents to the substrate, and thus allowing the cleavage of the substrate by intracellular elastases to begin. Accordingly, the microcontroller 106 powers the LED 112, waits 1 second for it to stabilize, and then captures the photosensor 118 reading of the light level transmitted through the reaction chamber (Step 14). This reading of light transmittance is taken as soon as the substrate is injected and mixed (Time 0), and then at 30 second intervals thereafter.

In a typical run, the light transmittance will cause the photometer to register 30,000 arbitrary units of light at the first reading (Time 0), and due to the cleavage of the elastase substrate, that level will decrease to 3,000 units by the end of the 10 minute reading interval. The 30,000 units is said to be 100% transmittance at time 0, then each subsequent reading is expressed as a percent of that baseline, such that 3,000 units would be 10% transmittance at the final time reading (Step 15). The % transmittance (% T) is converted to absorbance (A) as shown in Table 2 (Step 16), which is further analyzed to compute the slope of absorbance over time (Step 17), where this slope is said to be the maximum velocity of the elastase activity (Vmax) as detailed in Table 2 (Step 18 of Table 1).

TABLE 1
Steps in the CyBIS Process

| | Basic steps in the CyBis Process | | |
|---|---|---|---|
| 1 | Clean, dry and stick the finger to bleed | | |
| 2 | Collect blood in hematocrit tube | 50-75 | ul |
| 3 | Add 75 ul of BIBS buffer* and CD15 beads | | |
| 4 | Inject sample into reaction chamber of CyBIS | | |
| | block | | |
| 5 | Connect block to connectors for fluids | | |
| 6 | Activate software on microcontroller | | |
| 7 | Blood and buffer are mixed gently | 20 | min |
| 8 | Beads are captured by powering the | 1 | min |
| | electromagnet | | |
| 9 | Blood is aspirated and flushed with clean buffer | | |
| 10 | Magnet is released and buffer is agitated | | |
| 11 | Magnet is engaged to collect cells | | |
| 12 | Repeat 2X more | | |
| 13 | Suspend beads with cells in Substrate Buffer** | | |
| 14 | Read optical transmission at 405 nm each minute | 10 | min |
| 15 | Convert the transmission to % of baseline OD | | |
| 16 | Convert the % transmission to Absorbance | | |
| 17 | Compute the slope of the Absorbance overtime | | |
| 18 | Slope = Vmax of elastase activity | | |

### Typical Buffers for CD15 Isolation

Bead Isolation Buffer (BIB) for CD15+ PMN isolation: Hanks Balanced Salt Solution (HBSS) without Ca+2 or Mg+2+1% BSA, 0.5 mM EDTA

Washing buffer: 1× phosphate-buffered saline (PBS) without Ca+2 and Mg+2

Substrate buffer: 1× phosphate-buffered saline (PBS) with Ca+2 and Mg+2 pH 8.0 and 0.2% Triton X-100, which facilitates lysis of the cells and interaction between enzyme and substrate.

Calculating the Enzymatic Rate from the Transmission of Light Over Time

As shown in Table 2, the transmission of light through the reaction chamber is measured at 30 second or 1 minute intervals, by transiently powering the 405 nm LED, allowing it to stabilize and then reading the photometer values, with the reaction chamber light path between them. The digital values from the photometer are proportional to the light detected and decrease over time as the elastase enzymes cleave the colorless substrate into a pale yellow product that absorbs 405 nm light, thereby decreasing the signal received at the photometer. As detailed in lower portion of Table 2, the decreasing transmission of the violet light, detected in the ‘blue’ channel of the photometer, is converted to increasing absorbance by standard calculations. The slope of the absorbance overtime is the velocity of the reaction, and for the purposes of CyBIS, the rate over the first 5 minutes is taken to be the Vmax.

TABLE 2
Calculation of enzymatic rate from the transmission
of light through the reaction.

| Minute | Transmittance | % Transmittance | Absorbance | Slope |
|---|---|---|---|---|
| 0 | 2765 | 1.000 | 0.000 | |
| 1 | 654 | 0.237 | 0.626 | 0.626 |
| 2 | 176 | 0.064 | 1.196 | 0.598 |
| 3 | 55 | 0.020 | 1.701 | 0.567 |
| 4 | 19 | 0.007 | 2.163 | 0.541 |
| 5 | 7 | 0.003 | 2.597 | 0.519 |

| | |
|---|---|
| Average slope= | 0.570 |
| Vmax = slope in mOD/min= | 570.0 |

Calculation of Absorbance and Elastase Activity (Vmax)
The photometer measure transmission (T) of light at 405 nm
This is reported in arbitrary digital units on a scale of 0-65, 536
The Transmission at the time the enzyme substrate is added to the captured CD15+ cells is Time 0
% Transmission is calculated at each time by % T = T(time X)/T(time 0)
Absorbance is calculated as A = 2-LOG((100*% T), 10) which is 2-log10 of % T at each time.
The rate (velocity) of enzymatic cleavage of the substrate, which generates the absorbance is calculated as the average slope of Absorbance over the first 5 minutes of the reaction.
Example: (using purified elastase at a high concentration)

Diagnosis of Internal Infections from Elastase Activity

In the present examples, the elastase activity of CD15+ PMNs from a blood sample of a subject is used to aid in the diagnosis of an internal infection. There are 2 general strategies that can be employed to translate the CyBIS elastase activity into the risk of internal infection in a given subject:

(1) Historical reference data. In this strategy, when the exact conditions of the assay are defined, such as the amount of blood used, the temperature of the reactions, and substrate concentration, then a sufficient number of subjects without known internal infections are used to compose a reference set. For example, 20 subjects would be run on the CyBIS device and then the average Vmax would be computed. For a test subject to be examined by CyBIS, if their Vmax was >1.5-fold higher than that reference average, then they would be considered at risk of an internal infections and treated with other diagnostics or therapies, based on all of the additional evidence available to the healthcare professional. For example, at present, normal lab donors have an average Vmax of about 100 mOD/min, and thus, we would set a threshold for infection at 150 mOD/min in this approach.

(2) Concurrent reference data. In this strategy, a reference sample of artificial ‘cells’ would be manufactured such that it had CD15 on the surface of the bead, and elastase enzyme within or attached to the bead. This reference sample would be calibrated in such a manner that the reference sample would contain elastase activity comparable to a normal, uninfected subject (for example 100 mOD/min). A separate, parallel channel would be devised on the CyBIS block and device for this reference sample, and if the test sample from a patient was found to exhibit >1.5-fold higher elastase activity than the reference sample, then the patient would be considered at risk of infection. This approach has the advantage of incorporating built-in compensation for any unknown variations in the reagents or the conditions of the assay, such as temperature or atmospheric pressure.

Regardless of the reference data used, in both exemplary embodiments, the results of the assay are interpretable as a quantitative measure of the activity of the circulating neutrophils. Said differently, the CyBIS test is not a binary positive/negative test for internal infections. The higher the PMN elastase activity, the greater the likelihood of the presence of an infection. Importantly, the CyBIS elastase activity is not the SOLE indicator of the presence of an infection. It is designed to be evaluated by a healthcare professional as one parameter that is combined with other evidence about the subject to determine the risk of an internal infection. This other evidence would include the presence of a fever, malaise, pain, an elevated WBC, or an elevated neutrophil/lymphocyte ratio (NLR).

Data Generated from the Prototype Integrated POC CyBis Device

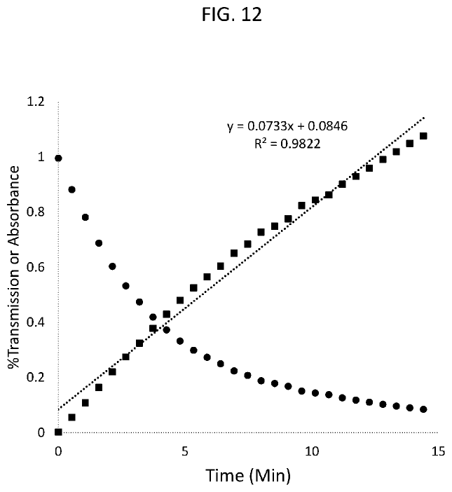

The exemplary CyBIS system, as described herein, was rendered to use and applied to small blood samples from laboratory donors (FIG. 12). The blood was collected by venipuncture, although similar results have been obtained from fingersticks of blood, and mixed with BIBS buffer and anti-CD15 magnetic beads (30 ul) and then applied to the reaction chamber in the CyBIS block. The CyBIS block is then connected to the CyBIS device, which gently mixes the blood and beads for 20 minutes, collects the magnetically captured cells, washes them free of RBC and plasma inhibitors, applies a lysis buffer containing colorimetric substrate, and then quantitates the transmission of 405 nm light through the chamber over a 20 minute period. The microcontroller in the CyBIS Device automatically converts the transmission at each time point to % transmission, and then absorbance, and then calculates the Vmax, as detailed in Table 2.

In the embodiment of FIGS. 9-11, the Device 101 includes a processing device, such as microcontroller 106, to perform various functions and operations in accordance with the disclosure. The processing device can be, for instance, a computer, personal computer (PC), server or mainframe computer, or more generally a computing device, processor, application specific integrated circuits (ASIC), or controller. The processing device can be provided with one or more of a wide variety of components or subsystems including, for example, wired or wireless communication links, input devices (such as touch screen, keyboard, mouse) for user control or input, monitors for displaying information to the user, and/or storage device(s) such as memory, RAM, ROM, DVD, CD-ROM, analog or digital memory, flash drive, database, computer-readable media, and/or hard drive/disks. All or parts of the system, processes, and/or data utilized in the system of the disclosure can be stored on or read from the storage device(s). The storage device(s) can have stored thereon machine executable instructions for performing the processes of the disclosure. The processing device can execute software that can be stored on the storage device. Unless indicated otherwise, the process is preferably implemented automatically by the processor substantially in real time without delay. And the processing device can be located locally in the Device 101, or remotely at a central location in communication with other processing devices.

To summarize, the Device 101, with the Block 108, has the effect of isolating particular cells of interest, in this case PMNs, and washing them free of irrelevant cells and proteins in blood that can interfere with the measurement of elastase activity. Once captured by the magnetic beads and electromagnet, the interfering cells and proteins are washed away, and the captured cells are lysed and exposed to an elastase substrate. Cleavage of that substrate by the lysed cells is a novel and unique measure of the functional activity of the PMNs. This combination of cell capture and kinetic enzyme assay is a sensitive measure of the number of PMNs and their content of active elastases. The rate of substrate cleavage is an excellent measure of the total elastase activity in the captured cells, and thus, reports the functional activity of these important immune cells in a given sample of a patient. Whereas the long-standing measurement of white blood cell (WBC) count is a crude, but accepted measure of infection, the CyBIS elastase activity is a more refined measure of not just WBC count, but of the net activity of white cells, especially PMNs.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All references cited herein are incorporated by reference.

1. An assay device for detecting immune cell activation comprising:
a reaction chamber receiving a blood sample and antibody coated magnetic beads;
a light source transmitting light through said reaction chamber;
an electromagnet aligned with said reaction chamber to capture a complex between certain cells in the blood sample and the antibody coated magnetic beads;
a photosensor receiving light passing through said reaction chamber, wherein a change in light transmittance due to cleavage of an elastase substrate is indicative of immune cell activation in the blood sample.

2. The device of claim 1, further comprising a bandpass filter, wherein the light passed through the reaction chamber passes through the bandpass filter to said photosensor.

3. The device of claim 1, wherein said light source comprises a light emitting diode that transmits the light at a wavelength of approximately 405 nm.

4. The device of any of claim 1, further comprising a first valve, said microcontroller configured to open and close the first valve to remove unbound cells and contaminating proteins from cells in said reaction chamber.

5. The device of claim 4, further comprising a second valve, said microcontroller opens and closes a second valve to add washing buffer to remove unbound cells and contaminating proteins from cells in said reaction chamber.

6. The device of claim 5, further comprising a third valve, said microcontroller opens and closes a third valve to add substrate-containing buffer to the cells captured from the blood sample in said reaction chamber.

7. The device of claim 6, wherein the light transmitted by the light source passes through a collimating lens to said reaction chamber.

8-9. (canceled)

10. The device of claim 1, wherein a change in light transmittance over time indicates elastase activity in the collected cells in said reaction chamber.

11. The device of claim 1, wherein the antibody coated magnetic beads comprise CD15 antibody covalently bound to the beads.

12. The device of claim 1, wherein the antibody coated magnetic beads are superparamagnetic.

13. The device of claim 1, wherein the cells are polymorphonuclear neutrophils.

14. A method for detecting immune cell activation in a blood sample comprising:
(a) mixing antibody-coated magnetic beads with the blood sample;
(b) introducing an enzyme substrate to the contents of cells from the blood sample which are complexed with the antibody-coated magnetic beads, where the enzyme substrate cleaves in the presence of a neutrophil enzyme resulting in a product which absorbs light at a specific wavelength; and
(c) measuring light transmittance at the specific wavelength through the enzyme substrate exposed to the contents of the cells, wherein a change in the light transmittance is indicative of immune cell activation in the blood sample.

15-30. (canceled)

31. A method of treating a patient diagnosed with an infection comprising treating the patient for an infection, wherein the patient is diagnosed as having an infection by (i) mixing antibody-coated magnetic beads with a blood sample from the patient; (ii) introducing an enzyme substrate to the cells from the blood sample which are complexed with the antibody-coated magnetic beads, where the enzyme substrate cleaves in the presence of a neutrophil enzyme resulting in a product which absorbs light at a specific wavelength; and (iii) measuring light transmittance at the specific wavelength through the enzyme substrate exposed to the cells, wherein a change in the light transmittance is indicative of immune cell activation in the blood sample; and upon identifying immune cell activation in the blood sample by observing elastase activity above preset normative values, then diagnosing the patient as having an infection.

32. The method of claim 31, wherein step (a) further comprises separating the cells in the blood sample which are complexed with the antibody-coated magnetic beads from the remainder of the blood sample by applying an electromagnetic field and washing away with a washing buffer the cells and proteins from the blood sample which are not complexed with the antibody-coated magnetic beads.

33. The method of claim 31, wherein the step of adding an enzyme substrate comprises (i) lysing the cells complexed with the antibody coated beads to form a lysate, and (ii) mixing the enzyme substrate with the lysate.

34. The method of claim 31, wherein the antibody is an anti-CD15 antibody.

35. The method of any of claim 31, wherein the treatment step comprises administering one or more antibiotics, antibiotics, antivirals, antifungals, antiparasitics, or any combination of any of the foregoing to the patient.

36. The method of claim 31, wherein the treatment step comprises administering one or more antibiotics to the patient.

37. The method of claim 31, wherein the enzyme substrate is an elastase substrate and the neutrophil enzyme is elastase.

38. The method of claim 31, wherein step (c) comprises transmitting the light through a collimating lens prior to passing through the enzyme substrate which has been exposed to the cells.

39. The method of claim 31, wherein the light is at a wavelength of approximately 405 nm and step (c) comprises measuring the light transmittance at thirty second intervals.
